(19) United States
(12) Patent Application Publication
(10) Pub. No.: US 2021/0085917 A1
(43) Pub. Date: Mar. 25, 2021

(54) Percutaneous Nephrostomy System

(71) Applicants: Hyunjean Kim, Rancho Palos Verdes, CA (US); Cristobal Ducaud, Miami, FL (US); Omar Vigar, Miami, FL (US); Elias Atri, Hollywood, FL (US); Molly Hulbert, Downey, CA (US); Mark Maguire, Miami, FL (US)

(72) Inventors: Hyunjean Kim, Rancho Palos Verdes, CA (US); Cristobal Ducaud, Miami, FL (US); Omar Vigar, Miami, FL (US); Elias Atri, Hollywood, FL (US); Molly Hulbert, Downey, CA (US); Mark Maguire, Miami, FL (US)

(21) Appl. No.: 16/994,631

(22) Filed: Aug. 16, 2020

Related U.S. Application Data
(60) Provisional application No. 62/888,571, filed on Aug. 19, 2019.

(51) Int. Cl. A61M 25/00; A61M 39/22

(52) U.S. Cl. CPC A61M 25/0017; A61M 2039/248; A61M 39/22

(57) ABSTRACT

A percutaneous nephrostomy catheter system includes a nephrostomy catheter lumen, an extension tubing and a collection bag. The extension tubing is inserted into the nephrostomy catheter lumen. The collection bag is fluidly coupled to the extension tubing. A percutaneous nephrostomy catheter system also includes a catheter hub which employs a Luer valve in order to facilitate nephrostomy care so that when the percutaneous nephrostomy system is in place urine flows from the renal pelvis into the nephrostomy catheter lumen, the extension tubing and the collection bag. The Luer valve employs a scalloped Luer-style male connector which, when screwed into the catheter hub allows flow.

### BACKGROUND OF THE INVENTION

### Field of the Invention

The present invention relates to devices and kits for percutaneous surgery access and more specifically to needle placement procedures and repairable devices that minimize or eliminate the use of fluoroscopy in order to reduce radiation exposure by decreasing time in the operating room.

The present invention also relates generally to indwelling drainage catheters and more particularly to a catheter that is configured to include spiral, helical or radial geometry on the external surface that allows the catheter to be introduced and locked into the anatomy via threading, linear indexing or similar action.

### Description of the Prior Art

Percutaneous nephrostomy is a common and well-known procedure in most Interventional Radiology practices. Typical indications include relief of urinary tract obstruction, urinary diversion, access for therapies, and diagnostic testing (1). The most common of these indications is relief of urinary tract obstruction, most frequently caused by stones, malignancy, and iatrogenic benign strictures.

From 2003-2012, the number of nephrostomy and nephrostomy-related hospital stays increased from 37,200 to 50,200, which is equivalent to an average 2.5% increase annually (2). The cost of the procedure varies depending on a patient's health insurance, the practice setting, and the cost of the manufactured equipment (3). Lowest reported cost of placement is $2,393, while the estimated national average is $5,620 (4), with replacements for dislodged or migrated catheters costing upwards of $800-$925 (5). Given the relative frequency of these procedures, nephrostomies amount to a $282 million impact annually. Nephrostomies are an incredible life-changing procedure and their financial footprint is notable for equipment manufacturers, hospitals, physician, healthcare staff, insurance companies and the patients. The procedure and devices used are not without unique complications which, given the significant mark nephrostomies have made in healthcare, need to be addressed.

A simple PubMed search using keywords nephrostomy tube and dislodge yields 35 articles exploring ways to improve the outcome of replacing damaged and dislodged nephrostomy tubes, as well as the nephrostomy procedure in general (6). Post-operative dislodgement of nephrostomy tubes occurs after 1-5% of percutaneous nephrostomy placements (7,8). A single emergency department in the United Kingdom reported 56 such cases in a 12-month period (9). Failed instrumentation and daily care of the tube and procedural site are not only nuisances, but they can impact patient health, possibly leading to sepsis if not treated in a timely manner (10). The urine collection bags are known to be uncomfortable. Patients constantly report an inability to sleep on their back or side due to pain from the nephrostomy tube, affecting sleep quantity and quality and leading to an overall decrease in quality of life for patients.

According to UpToDate, low sleep quality can lead to issues such as anxiety, depression, poor judgement, and workplace errors. Patients with chronic decreased sleep are found to do less of the activities they enjoy, be immunosuppressed, have decreased efficiency at work, increased BMI, and an increased risk for cardiovascular events (11).

Functionally, nephrostomy devices are composed of four basic components: the portion within the renal pelvis, the portion traversing the renal parenchymal and thoracoabdominal wall to the exterior, the hub/stopcock assembly, and the collection bag with extension tubing.

The portion within the renal pelvis drains urine from the renal pelvis into the tube lumen while anchoring the catheter in position. Most devices utilize a pigtail shape (retention) with multiple side holes (drainage). A retention suture, secured at the hub, assists in securing this pigtail shape. A stopcock is typically connected between the hub and the tubing for the collection bag to redirect or block flow during bag emptying/changes or catheter flushing. The two primary concerns that this invention aims to address are mechanical failure and patient comfort.

These devices can dislodge and break, which is a burden to hospitals, healthcare professionals and patients, as described above. Among possible alterations at the skin attachment site to make the device more secure is the idea of adding a waist belt instead of a leg band to hold the nephrostomy bag. In this way, the tubing associated with the device would have less risk of entanglement and decreased interference with daily life. The bag itself would not pull on the attachment site with ambulation. This intervention could prevent dislodgement and improve patient comfort.

The hub and stopcock are bulky pieces composed of hard plastic that can cause discomfort to the patient as described above, affecting their quality of life. To address this issue would be making the hub and the stopcock from a silicone material that is malleable and relatively soft. Alternatively, moving the hub/stopcock assembly closer to the actual drainage bag would allow the patient to place this uncomfortable piece in a less intrusive location away from points of compression. A way to make the catheter-bag connection and flushing apparatus not only less bulky, but also easier to use, to make significant improvements of existing nephrostomy devices by drawing inspiration from other designs, both in the medical field and beyond, rather than creating a new technology. Presented in this specification are several improvements to address the various issues of nephrostomy devices. A wide variety of diagnostic or therapeutic procedures involve the introduction of a device through a natural or artificially created access pathway. A general objective of access systems, which have been developed for this purpose, is to minimize the cross-sectional area of the puncture, while maximizing the available space for the diagnostic or therapeutic instrument. These procedures include, among others, a wide variety of laprascopic diagnostic and therapeutic interventional procedures.

Percutaneous nephrostomy is one type of therapeutic interventional procedure that requires an artificially created pathway. Percutaneous nephrostomy is a minimally invasive procedure that can be used to provide percutaneous access to the upper urinary tract. At first, percutaneous nephrostomy was used only for urinary diversion but now it may be used for more complex procedures such as stone extraction, integrate endo-pyelotomy, and resection of transitional cell carcinoma of the upper urinary tract.

In many percutaneous nephrostomy systems, a stiff guidewire is first placed into the renal collection system through the renal parenchyma and the ureter using fluoroscopic control. A second “safety wire” may be placed with a dual lumen catheter for maintaining the tract should the first wire become dislodged or kinked. Once guidewire control is established, a dilator sheath is used to create the tract and establish a rigid working lumen. An early technique involved advancing a flexible, 8 French, tapered catheter over the first guidewire to provide guidewire protection as well as a stable path for the placement of larger diameter dilators and sheaths. The larger diameter sheaths are sequentially advanced over the catheter and each other until an approximately 34 French-tract is established. The inner sheaths or dilators may then be sequentially removed such that the outermost sheath defines a working lumen. In this system, tract formation is accomplished by the angular shearing force of each subsequent sheath placement, which cuts a path through the tissue. Because axial pressure is required to advance and place each sheath, care must be taken to avoid kinking the tapered catheter and/or advancing the sheaths too far and perforating the renal pelvis. This technique also requires many steps. A more recent technique utilizes a balloon that is advanced over the first guide wire. Once in place in the renal pelvis, the balloon is inflated with a dilute contrast media solution to enlarge the tract. Once the balloon is inflated to a suitable diameter, a rigid sheath is advanced over the balloon. Advancing the rigid sheath over the balloon typically requires applying axial force to the sheath and rotation. The balloon may then be deflated and removed from the rigid sheath so that the rigid sheath may define a working lumen. In general, this technique is considered less traumatic than the previously described technique. Nevertheless, placement of the rigid sheath still involves angular shearing forces and several steps. Additional information regarding percutaneous nephrostomy can be found in McDougall, E. M., et al. (2002), Percutaneous Approaches to the Upper Urinary Tract, Campbell's Urology. 8th ed, vol. 4, pp. 3320-3357, Chapter 98. Philadelphia, Saunders. A need therefore remains for improved access technology which allows a device to be percutaneously passed through a small diameter tissue tract, while accommodating the introduction of relatively large diameter instruments.

U.S. Pat. No. 10,349,976 expandable percutaneous sheath, for introduction into the body while in a first, low cross-sectional area configuration, and subsequent expansion to a second, enlarged cross-sectional configuration. The sheath is maintained in the first, low cross-sectional configuration by a tubular restraint. In one application, the sheath is utilized to provide access for a diagnostic or therapeutic procedure such as percutaneous nephrostomy or urinary bladder access.

U.S. Patent Application Publication No. 2019/0321074 teaches a percutaneous access system including trocars, for accessing desired locations within a subject's body through the subject's skin or other tissues that are configured to minimize incision sizes are disclosed. Such a percutaneous access system includes a cannula and an obturator. The cannula includes a passageway with a tapered section and an expandable section at its distal end. The expandable section may include leaves that are configured to extend radially outward as an elongated instrument that has an outer diameter that exceeds a minimum relaxed inner diameter of the tapered section of the passageway is forced through the tapered section. Methods for using such a percutaneous access system, including medical procedures, are also disclosed.

Trocars have been employed in the medical field for many years. A trocar includes a cannula (a hollow tube), an obturator with a sharpened tip that extends through a channel of the cannula, and a seal between the obturator and the cannula. The sharp top of the obturator, when extended from a distal end of the cannula, is configured to form an incision, or opening, through a subject's skin or other tissues and, thus, to be introduced into a site of interest (e.g., a location through which a laparoscopic procedure is to be performed, such as a cavity, a blood vessel.) within the subject's body. Once the sharp tip of the obturator and the distal end of the cannula have been positioned at the location of interest, the obturator may be removed from the channel of the cannula and the laparoscopic procedure may then be performed through the cannula. The distal end of the cannula of a conventional trocar typically has a fixed outer diameter, which is usually consistent with the outer diameter of a remainder of the cannula. Thus, the size of the incision made by the obturator must accommodate the outer diameter of the cannula. In embodiments where the channel of the cannula must accommodate medical instruments with relatively large outer diameters, a cannula with a larger outer diameter is required and, thus, a relatively large incision must be made through the subject's skin or other tissues. Larger incisions are typically more unsightly than smaller incisions, take longer to heal than smaller incisions, result in an undesirable amount of scar tissue relative to the amount of scar tissue generated as a subject heals from a smaller incision, and pose a greater risk of infection from the procedure and as the subject heals from the procedure.

U.S. Patent Application Publication No. 2019/0105465 teaches a percutaneous ureteral catheter which is configured to be deployed in a urinary tract of a patient and which includes a proximal portion configured to pass through a percutaneous opening and a distal portion including a retention portion. The retention portion is configured to be deployed in a kidney, renal pelvis, and/or bladder of the patient. The retention portion includes one or more protected drainage holes, ports or perforations and is configured, when deployed, to establish an outer periphery or protective surface area that inhibits mucosal tissue from occluding the one or more protected drainage holes, ports, or perforations upon application of negative pressure through the catheter. The renal or urinary system includes a pair of kidneys, each kidney being connected by a ureter to the bladder, and a urethra for draining fluid or urine produced by the kidneys from the bladder. The kidneys perform several vital functions for the human body including, for example, filtering the blood to eliminate waste in the form of urine. The kidneys also regulate electrolytes (e.g., sodium, potassium, and calcium) and metabolites, blood volume, blood pressure, blood pH, fluid volume, production of red blood cells, and bone metabolism. Adequate understanding of the anatomy and physiology of the kidneys is useful for understanding the impact that altered hemodynamics other fluid overload conditions have on their function. In normal anatomy, the two kidneys are located retroperitoneally in the abdominal cavity. The kidneys are bean-shaped encapsulated organs. Urine is formed by nephrons, the functional unit of the kidney, and then flows through a system of converging tubules called collecting ducts. The collecting ducts come together to form minor calyces, then major calyces, which ultimately join near the concave portion of the kidney (renal pelvis). A major function of the renal pelvis is to direct urine flow to the ureter. Urine flows from the renal pelvis into the ureter, a tube-like structure that carries the urine from the kidneys into the bladder. The outer layer of the kidney is called the cortex and is a rigid fibrous encapsulation. The interior of the kidney is called the medulla. The medulla structures are arranged in pyramids. most Each kidney is made up of approximately one million nephrons. Each nephron includes the glomerulus, Bowman's capsule, and tubules. The tubules include the proximal convoluted tubule, the loop of Henle, the distal convoluted tubule, and the collecting duct. The nephrons contained in the cortex layer of the kidney are distinct from the anatomy of those contained in the medulla. The principal difference is the length of the loop of Henle. Medullary nephrons contain a longer loop of Henle, which, under normal circumstances, allows greater regulation of water and sodium reabsorption than in the cortex nephrons. The glomerulus is the beginning of the nephron and is responsible for the initial filtration of blood. Afferent arterioles pass blood into the glomerular capillaries, where hydrostatic pressure pushes water and solutes into Bowman's capsule. Net filtration pressure is expressed as the hydrostatic pressure in the afferent arteriole minus the hydrostatic pressure in Bowman's space minus the osmotic pressure in the efferent arteriole. Net Filtration Pressure=Hydrostatic Pressure (Afferent Arteriole)−Hydrostatic Pressure (Bowman's Space)−Osmotic Pressure (Efferent Arteriole). The magnitude of this net filtration pressure defined by Equation 1 determines how much ultra-filtrate is formed in Bowman's space and delivered to the tubules. The remaining blood exits the glomerulus via the efferent arteriole. Normal glomerular filtration, or delivery of ultra-filtrate into the tubules, is about 90 ml/min/1.73 m.sup.2. The glomerulus has a three-layer filtration structure, which includes the vascular endothelium, a glomerular basement membrane, and podocytes. Normally, large proteins such as albumin and red blood cells, are not filtered into Bowman's space. However, elevated glomerular pressures and mesangial expansion create surface area changes on the basement membrane and larger fenestrations between the podocytes allowing larger proteins to pass into Bowman's space. Ultra-filtrate collected in Bowman's space is delivered first to the proximal convoluted tubule. Re-absorption and secretion of water and solutes in the tubules is performed by a mix of active transport channels and passive pressure gradients. The proximal convoluted tubules normally reabsorb most of the sodium chloride and water, and nearly all glucose and amino acids that were filtered by the glomerulus. The loop of Henle has two components that are designed to concentrate wastes in the urine. The descending limb is highly water permeable and reabsorbs most of the remaining water. The ascending limb reabsorbs 25% of the remaining sodium chloride, creating a concentrated urine in terms of urea and creatinine. The distal convoluted tubule normally reabsorbs a small proportion of sodium chloride, and the osmotic gradient creates conditions for the water to follow. Under normal conditions, there is a net filtration of approximately 14 mmHg. The impact of venous congestion can be a significant decrease in net filtration, down to approximately 4 mmHg. See Jessup M., The cardiorenal syndrome: Do we need a change of strategy or a change of tactics, JACC 53(7):597-600, 2009 (hereinafter “Jessup”). The second filtration stage occurs at the proximal tubules. Most of the secretion and absorption from urine occurs in tubules in the medullary nephrons. Active transport of sodium from the tubule into the interstitial space initiates this process. However, the hydrostatic forces dominate the net exchange of solutes and water. Under normal circumstances, it is believed that 75% of the sodium is reabsorbed back into lymphatic or venous circulation. Because the kidney is encapsulated, it is sensitive to changes in hydrostatic pressures from both venous and lymphatic congestion. During venous congestion the retention of sodium and water can exceed 85%, further perpetuating the renal congestion. See Verbrugge et al., The kidney in congestive heart failure: Are natriuresis, sodium, and diruetucs really the good, the bad and the ugly? European Journal of Heart Failure 2014:16, 133-42 (hereinafter “Verbrugge”). Venous congestion can lead to a prerenal form of acute kidney injury (AKI). Prerenal AKI is due to a loss of perfusion (or loss of blood flow) through the kidney. Many clinicians focus on the lack of flow into the kidney due to shock. However, there is also evidence that a lack of blood flow out of the organ due to venous congestion can be a clinically important sustaining injury. See Damman K, Importance of venous congestion for worsening renal function in advanced decompensated heart failure, JACC 17:589-96, 2009 (hereinafter “Damman”). Prerenal AKI occurs across a wide variety of diagnoses requiring critical care admissions. The most prominent admissions are for sepsis and Acute Decompensated Heart Failure (ADHF). Additional admissions include cardiovascular surgery, general surgery, cirrhosis, trauma, burns, and pancreatitis. While there is wide clinical variability in the presentation of these disease states, a common denominator is an elevated central venous pressure. In the case of ADHF, the elevated central venous pressure caused by heart failure leads to pulmonary edema, and, subsequently, dyspnea in turn precipitating the admission. In the case of sepsis, the elevated central venous pressure is largely a result of aggressive fluid resuscitation. Whether the primary insult was low perfusion due to hypovolemia or sodium and fluid retention, the sustaining injury is the venous congestion resulting in inadequate perfusion. Hypertension is another widely recognized state that creates perturbations within the active and passive transport systems of the kidney(s). Hypertension directly impacts afferent arteriole pressure and results in a proportional increase in net filtration pressure within the glomerulus. The increased filtration fraction also elevates the peritubular capillary pressure, which stimulates sodium and water re-absorption. See Verbrugge. Because the kidney is an encapsulated organ, it is sensitive to pressure changes in the medullary pyramids. The elevated renal venous pressure creates congestion that leads to a rise in the interstitial pressures. The elevated interstitial pressures exert forces upon both the glomerulus and tubules. See Verbrugge. In the glomerulus, the elevated interstitial pressures directly oppose filtration. The increased pressures increase the interstitial fluid, thereby increasing the hydrostatic pressures in the interstitial fluid and peritubular capillaries in the medulla of the kidney. In both instances, hypoxia can ensue leading to cellular injury and further loss of perfusion. The net result is a further exacerbation of the sodium and water re-absorption creating a negative feedback. See Verbrugge, 133-42. Fluid overload, particularly in the abdominal cavity is associated with many diseases and conditions, including elevated intra-abdominal pressure, abdominal compartment syndrome, and acute renal failure. Fluid overload can be addressed through renal replacement therapy. See Peters, C. D., Short and Long-Term Effects of the Angiotensin II Receptor Blocker Irbesartanon Intradialytic Central Hemodynamics: A Randomized Double-Blind Placebo-Controlled One-Year Intervention Trial (the SAFIR Study), PLoS ONE (2015) 10(6): e0126882. doi:10.1371/journal.pone.0126882 (hereinafter “Peters”). However, such a clinical strategy provides no improvement in renal function for patients with the cardiorenal syndrome. See Bart B, Ultrafiltration in decompensated heart failure with cardiorenal syndrome, NEJM 2012; 367:2296-2304 (hereinafter “Bart”). In view of such problematic effects of fluid retention, a system and a method for improving removal of fluid such as urine from the patient and, specifically for increasing quantity and quality of fluid output from the kidneys, are needed. Percutaneous access is a commonly used step for the treatment and the testing of a variety of diseases and conditions in a plethora of surgical and clinical procedures. An initial step in many forms of percutaneous surgery is the insertion of a wire for later access into the inner portion of a lumen, space, viscous, or organ. This type of access could be placement of a needle through the skin into the kidney for access into one of the calyces of the kidney for removing kidney stones, such as in a percutaneous nephrolithotomy (PCNL) procedure. This step of the percutaneous procedure is often one of the most difficult steps and often requires real-time, imaging guidance with ultrasound, CT or fluoroscopy. Conventional techniques for needle placement in PCNL can require the use of continuous fluoroscopy during the insertion of the needle into the collecting system. Due to the depth of the tissues surrounding the kidney and the variation of the renal position caused by ventilation the surgeon is asked to hit a small moving target positioned deep inside the body and slight imprecision in needle positioning may lead to complete failure to access the desired space. Subsequently, surgeons are required to grasp a needle using either their hands by placing them directly inside the fluoroscopy beam or using a needle holder or device for holding the needle thereby decreasing their control and ability to perceive tactile subtle cues regarding tissue densities.

Fluoroscopy guidance accounts for a substantial percentage of the procedural radiation exposure to the patient as well as the surgical team. Every patient poses a different challenge and significant amounts of fluoroscopy can be used to navigate the trocar needle through the patient's anatomy. During needle placement, the amount of fluoroscopy required to obtain access is often several minutes and may be greater than sixty minutes of fluoroscopy time. Sixty minutes of fluoroscopy may be associated with significant radiation exposure and, depending upon the location of the fluoroscopy beam and the size of the patient, may exceed the recommended yearly occupational exposures of radiation. The deterministic effects of radiation occur quickly following exposure and may include sterility, cataracts, skin erythema and damage to the blood production system, intestinal function, or neurologic function.

In contrast, the stochastic effects of radiation are not directly dose dependent and may occur at any time following radiation exposure and may include genetic damage, cancer, and mental effects. High levels of radiation exposure have been recognized as a potential carcinogenic risk to the patient since the high-energy radiation may cause DNA mutation. It has been shown that a few minutes of fluoroscopy time at standard settings will confer a 1/1,000 risk of developing fatal cancer. For every 1000 patients exposed to even 10 mSv of radiation, one of those will develop cancer as a result. Further, fluoroscopy exposure is also known to have a cumulative effect over time, increasing the risk of stochastic effects on both the patient and the staff members, including the physician. As there is no safe lower limit (no safe threshold), below which no risk for cancer will occur and since higher the exposure the greater the risk, it is important to decrease the radiation exposure of patients during percutaneous access.

There is need for needle placement procedures and devices that minimize or eliminate the use of fluoroscopy in order to reduce radiation exposure. There is also need for devices and methods that would simplify surgical procedures and lower the costs associated with the same. Further, there is need for devices and methods of using the same that would reduce medical waste and the costs of disposal of this medical waste during and after a surgical procedure.

Flexible catheters are used for percutaneous drainage of an abscess or pocket of fluid in the body to the exterior by either gravity or negative pressure. Fluid collection may be the result of an infection, surgery, trauma, or other causes. Typical fluids include biliary, nephrostomy, pleural, urinary, and mediastinal collections. As an alternative to providing drainage, these catheters can also be used to introduce substances, such as fluids, into a patient's body.

In percutaneous drainage procedures, a catheter is typically introduced into a patient through a hypodermic needle or a trocar. A guidewire is inserted through the needle or the trocar, which is then removed. The catheter tube, with a stiffening cannula, then passes over the previously emplaced guide wire into the drainage site in the body cavity. The stiffening cannula is then removed.

Once a drainage catheter is in position in the body cavity, it is desirable to anchor the catheter before drainage begins. Typically, this can be done by forming a restraining portion in the distal end of the catheter in the form of a pigtail or “J-curve.” For a pigtail configuration, a flexible tension member, such as a suture thread, extends through draw ports at two spaced positions along the distal portion of the catheter. The restraining portion is conventionally activated by manually pulling the suture thread so that the two draw ports move toward each other as the pigtail loop forms at the distal end of the catheter. When the suture thread is taut, it prevents the pigtail loop from straightening by holding the juxtaposed portions of the catheter together in a locked position. The restraining portion is thus in a shape capable of resisting displacement from the body cavity. Once actuated, this restraining portion prevents removal of the catheter. When the catheter is ready to be removed, the cannula is inserted through the lumen until it reaches the pigtail loop. The restraining portion at the distal end is unlocked by cutting or releasing the suture at the proximal end, where the catheter protrudes from the body. Then the stiff cannula can be advanced distally to straighten the pigtail and help remove the catheter from the patient.

A preformed curve in the shape of a malecot rib has also been used as a possible anchoring mechanism. In this configuration, longitudinal slits are located in the restraining portion of the catheter at the distal end. The rib is activated in a similar manner as the pigtail configuration by manipulating a tension member, except the restraining portion is formed in the shape of multiple wings (typically two or four) instead of a pigtail. Successful procedures involving percutaneous drainage depend upon the initial placement of the drainage catheter and having the catheter remain in place for the duration of the treatment. Without adequate anchoring or support, catheter dislodging may result due to body movements by the patient or under other conditions. There are disadvantages of relying on a configuration such as the pigtail or the malecot rib as the sole anchoring mechanism. The actuation of a pigtail loop may not result in a precise placement because the pigtail has some compressibility and may migrate within the body cavity, causing movement at the proximal end of the catheter near the incision area as well. Due to the uncertainty of placement, additional steps may be necessary to confirm that the restraining portion has been actuated. Another potential problem relates to the structure of the pigtail. As the anchoring mechanism preventing the inadvertent removal of the catheter, the pigtail is constantly subject to forces pulling against it. It is possible for a pigtail restraining portion to give way and collapse on itself. Such a collapse would destabilize the location of the catheter and adversely affect drainage. Additionally, the pigtail may be difficult to form or engage in small collection pools or may float in larger collection pools.

U.S. Pat. No. 6,971,390 teaches a catheter connection and repair system which effects fluid-tight coupling and mechanical joinder between a medical device and a catheter tube. An elongated catheter connection stem attached to the medical device is received in the lumen of the catheter. A catheter securement collar is advanced proximally along the exterior of the catheter and connection stem inside the catheter into the assembled condition of the connection system, passing a locking ring on the interior of the securement collar over an enlargement on the distal end of the connection stem. Maximum catheter compression occurs at the locking ring, while a void internal of the catheter system is filled with an enlarged securement lip of catheter material. Distal of maximum catheter wall compression, the catheter traverses a tortuous path between the locking ring and the enlargement, around the shoulder of the enlargement, and along the adjacent surface of the enlargement.

Systems for securing medical catheters to medical devices that accommodate fluid flow are under continual redesign and refinement undertaken for the purposes of improving reliability, increasing ease of assembly, and accommodating for the development and adoption by the medical industry of new materials for medical catheters. U.S. Pat. No. 6,651,956 teaches a slit type, swabable valve which includes a stem having a slit at an end thereof. The valve stem is in a valve body and is deformable. When a tip of an instrument is engaged with a slit in the stem, the stem shifts in the valve body, top portion folds inward, the slit seals against the instrument and allows liquid to flow through the stem, to or from the instrument. All components have circular cross-sectional geometry. Presently, there are many types of swabable valves designed to address needlesticks safety issues. Such valves must satisfy many requirements. They must safely withstand, without loss of performance, at least 100 connects and disconnects to an injection site before the set is replaced. In addition, that connection shall be maintained for an extended time-period before disconnection is made. Still, the site shall be capable of accepting subsequent connections without allowing any leakage. Such valves must seal against pressurized fluid within a set. They must withstand pressures in excess of 25 psi, for a short time, such as during an injection made through an adjacent site or if a pump is nearby. Such valves shall not contain any dead space where fluid can collect and not be readily flushed away. Also, priming volume should be minimized. They must also be easily accessible by standard luer connectors and provide secure locking features, so such connectors could be left connected to the site without further assistance from a practitioner. Such valves shall be manufactured at high speeds and low cost. At the same time, the design must allow for minimal manufacturing defects. It is desirable that such valves have as few components as possible, and be easily assembled, without requiring any difficult component orientation or positioning. Another highly desirable feature is easy and safe swabability of the valve inlet area. Most current valves restrict free flow of passing fluid by employing narrow passages, ribs or internal cannula-like features. Restricting the flow path in such a manner may create conditions for hemolytic damage. Such restrictions also make the valve generally more difficult to flush. Some other valves are shown in U.S. Pat. Nos. 6,325,782 and 6,290,206. Some valves' stems or seals employ an opening that is closed upon assembly. This opening must be produced during molding and requires a core pin to extend all the way through the part, creating a possibility for flash to develop at the core pin shut off area. Such flash would then be found at the proximal end of the stem and present a possible danger if removed by the action of a penetrating luer connector as this would case the flash to be pushed into the fluid flow path.

Valves that employ resilient stems with a slit have an elliptical cross-sectional geometry and slit orientation must be precisely controlled by positioning system during slit manufacture or during assembly. Other types of valve are shown in U.S. Pat. Nos. 6,050,978 and 5,354,275. In medical applications, it is usually desirable to prevent the patient from being exposed to the fluid which is being injected to or extracted from the patient, and it is desirable to insulate nurses and doctors from exposure to the liquid which may contain the patient's blood or waste products. Often the instrument used to inject or withdraw the fluid (which is generally the male component of the syringe), retains some of the fluid on the tip thereof, thus providing a risk to nurses and doctors of being exposed to the fluid. Wiping off this fluid prior to disconnecting the instrument is highly desirable. Some similar devices currently on the market employ thin ribs or cannula-like housing details, which might be susceptible to breakage. Such breakage could damage the flexible sealing stem in the valve, or the flash could become loose inside the flow path. The same ribs or narrow housing channels present obstacles for smooth fluid flow, thus restricting flow and, in the case of blood transfer, they increase the risk of mechanical hemolytic damage.

One in five patients admitted to hospitals receive an indwelling urinary catheter (hereinafter, “catheter”). When a patient receives a catheter, and the longer the catheter is placed inside the body, the more likely the patient will develop a urinary tract infection (UTI). Hospital-associated infections (HAIs) are infections acquired during the course of receiving treatment for other conditions within a hospital setting. In the United States according to the Centers for Disease Control and Prevention (CDC) more than four million patients develop HAIs in the United States each year with about 99,000 of such cases resulting in death. UTIs may be caused by microbes (e.g. bacteria) entering the body through the catheter. The distribution of microbes among patients with hospital-acquired urinary tract-related bloodstream infections may include enterococcus, candida, E. coli, klebsiella, staphylococcus, and the like. According to the ICHP, the urinary tract is the most common site of HAIs and accounts for more than 64% of catherized patients in acute-care hospitals. In 2014, the CDC released a HAI Progress Report stating that among five categories of HAIs, only catheter-associated urinary tract infections (CAUTI) had an increase of incidents. Due to the increase of CAUTIs, the Centers for Medicare and Medicaid Services (CMS) will not allow hospitals to be reimbursed for treating a hospital acquired CAUTI because nosocomial CAUTIs and are believed to be “reasonably preventable.” The cost of treating UTIs could cost U.S. hospitals between $1.6 billion and $7.39 billion annually in lost Medicare reimbursements since treating each CAUTI incident may cost between US$600 to US$2,800 to treat. Microbes may travel intraluminally in the urinary system at least two ways: (1) suspended and floating microbial cells within the urine (i.e., bacteriuria) where there is backflow (reflux) of infected urine; and (2) by biofilm migration (i.e., colonies of microbial cells that may form layers and attach themselves to surfaces) that may ascend up through the inside of urinary tubing surfaces and into the catheter and potentially enter the patient's body. Research currently suggests that preventing urine backflow (reflux) from entering the catheter and/or incorporating a hurdle or barrier that prevents biofilms from ascending through the urinary collection system would help to mitigate the number of UTIs. Currently, the number of patients developing bacteriuria or UTIs after two and three days is 10% to 30%; while after one week or longer is near 90%, and; long-term catheterization (of one month or more) results in near 100% of patients with bacteriuria or UTIs. Although not all CAUTIs may be prevented, it is believed by the medical community that a substantial number of CAUTIs may be avoided by the proper management of indwelling and external catheters. CMS developed a list of recommendations and guidelines to help reduce UTIs and CAUTIs. One of these recommendations included preventing backflow (reflux) of urine which could contain bacteriuria or other microbes. Prevention may be the best way to manage nosocomial UTI, as opposed to focusing on expensive treatment and medicine, which may or may not be effective, as many microbes are becoming increasingly less sensitive and more resistant to antibiotics. A first and second anti-reflux barrier (e.g., one or more check-valves and/or one or more clamps) within a urinary system (e.g., including extension tubing and one or more check-valves) may be beneficial by denying microbes various routes of entry into the patient. If such a system is breached (e.g., by inappropriate opening), then microbes may enter the extension tubing and catheter and travel up into the urethra or body wall of the patient and infect the patient.

The inventors are not aware of any prior art that specifically addresses a device or component that forms an anti-reflux extension tubing system or where such a device or component may prevent urine backflow (reflux) of urine located within urinary extension tubing that connects the urinary indwelling and/or external catheter to the urine bag, or where such a device or component may prevent biofilm migration. Reference should also be made that there are no commercial products currently available that specifically addresses prevention of urine backflow in urinary collection systems, such as the extension tubing, by placing a check-valve inside the urinary extension tubing or within a connector of the extension tubing. Rather, unrelated prior art consists of inventions for closed-system and anti-reflux system are irrigation connectors, intravenous syringe ports, closed adapters for enteral formula delivery, and needleless IV access ports for small bore luers—i.e., none of this prior art deals with urinary systems. Some such prior art includes: preventing backflow for blood and urine specimens (i.e., not urinary collection system) and maintaining a closed-system for irrigation; and of using check-valves in feeding tubes. Such prior art does not incorporate any anti-reflux check-valve within the primary urinary extension tubing running from the indwelling catheter to the urine bag. The prior art may include a check-valve located within a urine bag. This prior art may prevent urine backflow (reflux) from the urine bag into the extension tubing. However, the urine collection system between the urine bag and the patient is vulnerable and presently has no barrier against urine backflow. If there is no anti-reflux valve (i.e., check-valve) present before the catheter to prevent urine backflow, then microbes in the urine may travel from the urine in tubing into the catheter leading to a CAUTI. There is a need in the art for satisfactorily addressing and reducing the high percentage of UTIs and CAUTIs that occur with current indwelling and/or external urinary catheter use. Many patients experience the development of a stricture or blockage within the ureter of one or both kidneys. The ureter is the muscular tube that connects the kidney to the bladder. As urine is made by the kidney it drains into a central collecting system of the kidney and then travels though the ureter into the bladder. Patients can develop strictures, or blockages, of the ureter due to kidney stones, cancers, infections, trauma, and prior medical instrumentations. In rare instances, some children are born with blockages of one or both ureters. If untreated, the blockage will eventually lead to kidney failure. Regardless the cause, the treatment for a blocked ureter is to relieve the blockage. Blockage removal is performed by inserting a long tube to connect the collecting system of the kidney to the bladder. This tube is called a stent and is placed through the ureter. Stent insertion is typically performed by one of two methods. The stent may be inserted urologically. With this method, a scope is advanced through the urethra into the bladder. A wire is then inserted into the ureter in a retrograde fashion, using the scope to thread the wire. When the wire reaches the collecting system of the kidney, a plastic stent is inserted over the wire. The stent is a straight plastic tube that has a pigtail-shaped curl on each end. Once in place, the wire is removed, and the scope is taken out of the bladder. One pigtail curl of the stent resides in the collecting system of the kidney and the other resides in the bladder. The straight portion of the stent traverses the ureter. This is performed using direct visualization with the scope and with fluoroscopic guidance. The stent usually stays in for a period of approximately three months, at which point the stent is then swapped out for a new stent by the urologist using a similar technique. The second method for insertion is to insert the stent percutaneously. This method is typically performed in stages. The right or left flank of the patient is sterilely prepared depending upon which kidney is to be accessed (sometimes both are accessed to treat bilateral blockages). Intravenous sedation is used. A smallbore needle is used to puncture the collecting system of the kidney and contrast is injected allowing the complete visualization of the entire collecting system. The central portion is initially punctured with a small needle, and then a larger needle is used to puncture a smaller but safer area of the collecting system. A guidewire is threaded into the collecting system of the kidney and a pigtail drain, or nephrostomy catheter, is placed, sutured to the back, and hooked up to a bag for external drainage. Once the urine has cleared from bleeding, the patient is brought back to the angiography table, placed prone, and a wire is inserted through the catheter into the kidney. The catheter is then removed. The wire is threaded through the ureter into the bladder (across the stricture) and a nephron-ureteral catheter is placed. The nephron-ureteral catheter is a long plastic tube that goes from the outside of the patient into the kidney's collecting system, through the ureter, and into the bladder. The catheter allows drainage of urine into the bladder and externally into a bag. The catheter typically stays in the patient for 7-10 days, at which time the patient is brought back to the angiography table and a wire is threaded through this tube into the bladder. The tube is removed, and an internal stent is placed using fluoroscopic guidance. This is the same type of stent that is placed by the urologist working through the bladder. This can be a complex and difficult procedure.

U.S. Pat. No. 10,405,943 teaches a kit which performs a reduced radiation percutaneous procedure. The kit includes a needle access device having a needle connected to a hub portion having an opaque cap portion, a non-opaque body portion positioned between the opaque cap portion and the needle and a channel extending through the opaque cap portion; a sticker having an adhesive side adapted to adhere to the skin of a patient, and a display surface opposite the adhesive side configured to enhance visualization of the sticker in low light; and a guidewire having a floppy portion with a distal end, an intermediate region connected to the floppy portion, such that the intermediate region is less floppy than the floppy portion; and an ultrasonic-profile-enhancing feature disposed within 3 centimeters of the distal end of the floppy portion.

U.S. Patent Application Publication No. 2012/0265173 teaches an indwelling drainage catheter which is configured to include spiral, helical or radial geometry on the external surface that allows the catheter to be introduced and locked into the anatomy via threading, linear indexing or similar action.

U.S. Pat. No. 10,610,677 teaches a connector-with-integrated-check-valve for minimizing microbial migration to catheter-tubing which is formed from three parts. A first part is a connector-for-catheter-tubing that is hollow and with an internal valve seat. A second part is an elastomer disc shaped gate. A third part is a connector-for-extension-tubing that is hollow and with support-surfaces. When one end of the connector-for-catheter-tubing is attached to one end of the connector-for-extension-tubing, a pocket is formed so that the valve seat is disposed opposite and facing the support-surfaces. The gate is disposed within this pocket such that when the gate contacts this valve seat due to urine backflow (reflux), the connector-with-integrated-check-valve is closed to such urine backflow. A remaining end of the connector-for-catheter-tubing is attachable to catheter-tubing and a remaining end of the connector-for-extension-tubing is attachable to the extension-tubing so that there is a continuous urine flow path from the catheter-tubing to the connector-with-integrated-check-valve, when open, and to the extension-tubing.

U.S. Pat. No. 10,695,161 teaches a nephron-ureteral catheter which includes a detachable portion such that when the detachable portion is removed, the catheter converts into an internal stent. The catheter includes a tube with two retention features, a detachable portion, and an inner tube. The inner tube is removably insertable into both the tube and the detachable portion, and a wire extends through at least a portion of a lumen of the inner tube and through the tube, to keep the pieces attached. The wire may be removed to remove the inner tube and then the detachable portion from the tube. When the detachable portion is attached to the tube, the catheter is a nephron-ureteral catheter. When the detachable portion is removed from the tube, the catheter becomes a stent.

Nephrostomy is the creation of a communication between the skin and kidney to provide for nephrostomy tube insertion. The objective in nephrostomy tube creation is to have the wire from outside the flank directed down the ureter to provide therapeutic drainage of an obstructed system. This allows for subsequent dilation of the tract, such as by a nephrostomy dilating balloon, between the kidney and the skin over a wire that extends down the ureter. The catheter and tract can also be used to facilitate stenting of a narrowed ureter or removal or treatment of stones obstructing the ureter. Current nephrostomy tube creation is dependent on x-ray exposure to guide the physician where to locate the nephrostomy puncture wire tract. There are currently two widely used techniques for nephrostomy tube creation. One technique utilizes an antegrade approach. The antegrade approach holds increased bleeding risk due to the puncture needle puncturing the interlobar arteries as it passes into the collecting system. This antegrade approach is also skill intensive because it requires advancing from the flank to an “unknown” calyx. In fact, studies have shown that recent urology resident graduates often do not continue to perform the antegrade nephrostomy technique after graduating due to difficulty of this procedure. The procedure also requires a relatively large amount of radiation exposure. The other technique commonly utilized is the Lawson technique. This technique is used to create a nephrostomy tract in a retrograde fashion. The Lawson technique is performed under fluoroscopy utilizing a deflecting wire inside a ureteric catheter to select the renal calyx to be entered. That is, fluoroscopy is used to identify the renal calyx for nephrostomy access. The Lawson technique is described for example in Smith's Textbook of Urology, 2007, BC Decker Inc., “Retrograde Access” by Dennis H. Hosking and is commercially available by Cook Urological, Inc. as the “Lawson Retrograde Nephrostomy Wire Puncture Set.” In the Lawson technique, a stainless steel 145 cm long guidewire (0.038 inches in diameter) having a 3 cm flexible tip is passed retrograde up the ureter into the renal pelvis under fluoroscopy. A 7 Fr catheter is passed over the guidewire into the renal pelvis and the guidewire is removed. A J-tipped wire in certain instances might be used to facilitate passage past an obstruction. Then the surgeon selects the optimal calyx for nephrostomy placement, optimization usually being defined by allowing easiest access to the renal calculi and the shortest tract. Once the calyx is selected, the 0.045 inch diameter deflecting wire guide is inserted through the lumen of the catheter and twist locked to the proximal end of the catheter. Deflection of the wire tip deflects the tip of the catheter, and the catheter and attached wire can be advanced into the selected calyx. It is recognized that due to obstructions, e.g. presence of calculi, it may not be possible to advance the catheter into the optimally desired calyx and consequently a less optimal calyx is selected by the surgeon. After insertion of the catheter into the selected calyx, the deflecting wire guide is removed from the lumen of the catheter, while maintaining the inner-calyx position of the catheter tip. A puncture wire and sheath as a unit are inserted through the catheter lumen, with the puncture wire sharp tip shielded by the sheath. During insertion through the catheter, the wire remains retracted within the sheath, and locked to the sheath by a pin vise lock, so its puncture tip is not exposed. The puncture wire and sheath are connected/locked to the proximal portion of the catheter. The puncture wire is then unlocked from the sheath, by untwisting the cap of the pin vise actuator to loosen the vise pin grip on the puncture wire, and then incrementally advanced from the distal end of the sheath through the flank, fascia and skin. After puncturing the skin, the puncture wire is advanced from below until approximately 15 cm of wire is externally visible. The pin vise lock securing the puncture wire to the insulating sheath is then re-locked. A fascial incising needle may or may not be passed over the puncture wire at the flank to incise fascia, and is then removed. As the 7 French catheter is advanced through the cystoscope below, the puncture wire is drawn further out of the flank, until the tip of the 7 French catheter is delivered out of the flank. At this time, the 7 French catheter is unlocked from its connection to the puncture wire assembly, and the puncture wire and insulating sheath are removed from below. A 0.038″ guidewire is then passed antegrade through the 7 French catheter from the flank, until it emerges out the lower end of the 7 French catheter at the cystoscope end. With this wire ‘through and through’ the body, the cystoscope and 7 French-catheter are removed, leaving the guidewire in place.

The retrograde Lawson approach has several advantages over the antegrade approach including providing the surgeon an anatomic approach to the renal pelvis, increased likelihood of avoiding the interlobar arteries during puncture, and inherently having a wire down the ureter, an important step in securing control over the nephrostomy tract. It is also less skill intensive, due in large part to the fact that it enables travel from the “known kidney” to the “unknown flank/skin,” which better respects the principles of surgery. Despite its advantages over the antegrade approach, there are several disadvantages to the Lawson technique. First, although requiring less radiation exposure, the patient is oftentimes still exposed to harmful doses of radiation. Secondly, it is often difficult to navigate the ureteric catheter beyond large obstructive stones in the renal pelvis. This inability to direct the catheter to the desired site (calyx) often leads the surgeon to access a less optimal calyx. Thirdly, fluoroscopy provides only a two-dimensional view of the renal anatomy, thereby limiting the ability to confidently select the calyx for tract dilation. Sometimes, there is even uncertainty as to which calyx is actually chosen due to the limited visibility provided by fluoroscopy. Consequently, it would be advantageous to provide a system and method that enables more precise calyx location, improves access to the calyx of choice, improves visualization, permits “fluoroscopy-free” calyx selection, and allows for preliminary laser lithotripsy of a portion of a stone that may block access to calyx of choice for nephrostomy creation. Also, of significance is that nephrostomy tube creation procedures are usually performed by interventional radiologists, which can further compound the risks and problems since urologists usually have better success rates for selecting the calyx for such procedures. It would be advantageous if such improved system and method could be more commonly performed by urologists. In an attempt to address some of the disadvantages of the Lawson technique, Dr. Larry C. Munch in an article entitled “Direct-Vision Modified Lawson Retrograde Nephrostomy Technique Using Flexible Ureteroscope” and published in the Journal of EndoUrology, Volume 3, Number 4, 1989, described a technique utilizing a flexible ureteroscope.

In this “Munch technique,” a flexible steerable ureteroscope was utilized to inspect the renal pelvis and calices. As described, a flexible cystoscopy is performed and a 0.035-inch, 145 cm guidewire is passed into the ureteral orifice. Position within the ureter is assessed with fluoroscopy. The cystoscope is removed and a ureteral access sheath with its obturator is advanced over the guidewire, and the obturator is then removed and the ureteroscope is passed through the sheath into the renal pelvis. An appropriate calyx is chosen visually, and then the 0.0017 inch-Lawson puncture wire and protective 3 Fr radiopaque Teflon sheath is passed through the working channel of the ureteroscope. The calyx is entered and the sheath embedded in the wall of the calyx, and then the pin-vise lock which locks the puncture wire and sheath together is opened and the puncture wire is advanced through the skin under visual and fluoroscopic control. The puncture wire protective sheath and ureteroscope are then withdrawn, leaving the puncture wire and ureteral access sheath in place. At the skin, an 18-gauge needle is passed over the puncture wire into the kidney and then removed. A 9 French fascial dilator is then passed over the 0.017 inch puncture wire into the kidney, where after the puncture wire is removed and a 0.038 inch guidewire is passed through the 9 French dilator until it passes down the ureter through the access sheath, and exits through the urethra. Although the Munch technique solves some of the problems associated with the Lawson technique, it is deficient in several respects. First, the Munch technique leaves the puncture wire exposed to the ureteropelvic junction. This creates the risk of cutting inside tissue, especially at the ureteropelvic junction, across which the very thin puncture wire passes. Tension on the puncture wire at the time of passing the antegrade exchange catheter may result in) internal ‘slicing’ of the ureteropelvic junction by the thin puncture wire. Second, at the time of deployment of the puncture wire, the Munch technique fails to secure the wire assembly and ureteroscope, forcing either the surgeon or an assistant to devote two hands to opening the pin-vise lock and advancing the puncture wire, all while holding the flexible ureteroscope in position in a selected calyx. This makes wire deployment cumbersome for the surgeon, less likely to be successful, requiring more skilled assistance, and increases the chances the tip of the flexible cystoscope will move out Of a selected location for nephrostomy creation. Third, Munch's technique of antegrade wire exchange is ineffectual and risks cutting the puncture wire with passage of 18 gage hollow bore needle over the wire. After passage of this needle, a 9 French fascial dilator is passed over the 0.017″ puncture wire, representing a wire-catheter mismatch which can result in tearing of internal tissues. This large jump from an 18-gauge needle to a 9 French fascial dilator is also cumbersome and has a high chance of failing to grant access to the kidney. Consequently, it would be advantageous to provide a system and method that would enable urologists to more economically and efficiently perform the nephrostomy procedure to obtain access for nephrostomy tube creation. Such procedure would have the above-noted advantages over the Lawson technique, e.g. improving calyx access, visualization etc., while also providing the advantages of reducing the number of surgical steps and securing the position of the components and protecting the puncture wire, especially at the ureteropelvic junction, thereby providing advantages over the Munch technique.

U.S. Pat. No. 8,888,787 teaches a percutaneous renal access system for creating a tract in retrograde fashion for nephrostomy tube creation and method which includes the steps of providing a puncture wire having a tissue penetrating tip shielded in a sheath, inserting the puncture wire and sheath through a channel in an ureteroscope, advancing the puncture wire from the sheath while visualizing under direct vision a position of the puncture wire, advancing the puncture wire through a selected calyx, and inserting antegrade a coaxial catheter over the puncture wire.

U.S. Pat. No. 10,405,943 teaches a kit which performs a reduced radiation percutaneous procedure. The kit includes a needle access device having a needle connected to a hub portion having an opaque cap portion, a non-opaque body portion positioned between the opaque cap portion and the needle and a channel extending through the opaque cap portion; a sticker having an adhesive side adapted to adhere to the skin of a patient, and a display surface opposite the adhesive side configured to enhance visualization of the sticker in low light; and a guidewire having a floppy portion with a distal end, an intermediate region connected to the floppy portion, such that the intermediate region is less floppy than the floppy portion; and an ultrasonic-profile-enhancing feature disposed within 3 centimeters of the distal end of the floppy portion.

U.S. Patent Application Publication No. 2012/0265173 teaches an indwelling drainage catheter which is configured to include spiral, helical or radial geometry on the external surface that allows the catheter to be introduced and locked into the anatomy via threading, linear indexing or similar action.

U.S. Pat. No. 10,722,693 teaches an inflatable device including a surface which has a network of polymer chains and which is configured to be inflatable into a therapeutically or diagnostically useful shape with at least one ultrashort laser pulse-formed modification in the surface. The network can include a network morphology that is substantially unchanged by modification with the ultrashort pulse laser. Ultrashort laser pulses can be laser pulses equal to or less than 1000 picoseconds in duration. Advantageously, the etching process uses a relatively low-heat laser to avoid significant heating of surrounding polymers while modifying the surface (and other structures) of the device. The process is configured so that the polymer chain morphology adjacent the modification is substantially unaffected by the low-heat laser. The resulting inflatable device has customized surface features while still retaining substantially homogenous polymer network morphology. This preserves the elasticity, especially the surface elasticity, of the inflatable device. Inflatable devices are used in many surgical and minimally invasive surgical (MIS) techniques and settings. Medical balloons with thinner walls, higher strength, and smaller profiles are designed to withstand high inflation pressures and are well suited for use in a broad range of diagnostic and therapeutic procedures. They can be produced in a variety of lengths, diameters, and shapes, including complex custom shapes for specific applications, and supplied with specialty coatings for added performance. In a typical MIS procedure, uninflated devices are positioned within an anatomical space and then filled with air or fluid to expand the device and possibly the anatomical space itself. This procedure is used to deliver a prosthetic heart valve or stent to a cardiac or vascular structure. Alternatively, an inflatable device can be used to dilate an anatomical structure, as in angioplasty procedures. Other surgical procedures that incorporate the use of inflatable devices include kyphoplasty, nephrostomy, gastric balloon placement, endometrial ablation, laparoscopic hernia repair and renal denervation. Inflatable devices can be used in the obstruction, dilation and/or stent placement within the following anatomical structures: sinuses, intestines, lacrimal ducts, Carpal tunnels, Eustachian tubes, the uterus, ureters, bile ducts, the trachea, the esophagus, the urethra, and the nasal passages. Inflatable devices are used in transcatheter aortic heart valve delivery procedures. In this procedure, a guidewire is delivered through the femoral artery, through the patient's vasculature to the native aortic valve, and placed within the left ventricle. A first balloon mounted on a catheter is inserted over the guidewire into the aortic valve and inflated to widen the structure. The first balloon is removed back down the guidewire. A second, folded balloon carrying the new prosthetic heart valve is delivered to the patient's diseased aortic valve. Alternatively, a prosthetic heart valve may be moved onto the balloon once it is inside the patient's body. Once positioned, the folded balloon is inflated, and the previously crimped valve is expanded to its full diameter. At its full diameter, the stent is lodged within the native heart valve. The second balloon is deflated and is routed back down the patient's vasculature and out the femoral artery via the guidewire, leaving the new valve in place.

The inventors hereby incorporate the above-referenced issued patents and published patent application publications into this specification.

### SUMMARY OF THE INVENTION

The present invention is generally directed to a percutaneous nephrostomy catheter system which includes a nephrostomy catheter lumen, an extension tubing inserted into the nephrostomy catheter lumen, a collection bag fluidly coupled to the extension tubing and a catheter hub.

In a first aspect of the present invention the percutaneous nephrostomy catheter system provides refinements in its design which improves patient is comfort/user friendliness.

In a second aspect of the present invention the percutaneous nephrostomy catheter system provides refinements in its design which enables simple, lower risk and inexpensive clinic or bedside repair in the case of fracture of the external catheter.

In a third aspect of the present invention the valve employs a scalloped Luer-style male connector which, when screwed into the catheter hub, allows urine flow.

Other aspects and many of the attendant advantages will be more readily appreciated as the same becomes better understood by reference to the following detailed description and considered in connection with the accompanying drawing in which like reference symbols designate like parts throughout the figures.

The features of the present invention which are believed to be novel are set forth with particularity in the appended claims.

### DESCRIPTION OF THE DRAWINGS

FIG. 1 is a schematic drawing of a novel retention system that employs coaxial sleeves with radial deployment of the distal outer sleeve for renal anchoring mechanism, OTW=over the wire.

FIG. 2 is a schematic drawing of an end cap needleless connector and two-piece connector represent complete hub assembly from a standard venous access catheter repair kit.

FIG. 3 is a schematic drawing of a four-step procedure for attaching Medcomp two-part adaptor to catheter tubing.

FIG. 4 is a schematic drawing of a Luer-activated needleless valve which connects to hub of nephrostomy catheter.

FIG. 5 is a schematic drawing of a cross-sectional diagram along the long axis of the device in “anchored” configuration demonstrating the two-part hub securing the coaxial sleeves in relative position.

FIG. 6 is a schematic drawing of a cross-sectional diagram along the long axis of the device in anchored configuration. 1) outer coaxial sleeve, continuous with anchoring ribs at the distal, intra-corporal end of the catheter; 2) inner coaxial sleeve, continuous with the drainage side-holes at the distal end of the catheter; 3A) male-threaded inner component of hub, containing a circumferential slot within which the inner sleeve is inserted as well as a beveled outer surface; 3B) female-threaded outer component of hub, which serves as a lockring securing the two coaxial sleeves in relative position.

FIG. 7 is a schematic drawing of a conceptual prototype. Several longitudinal slits were created in the distal portion of an 8-French vascular sheath, representing the outer coaxial sleeve. An 8-French all-purpose drainage catheter (lighter shade of blue) was inserted within the lumen of the vascular sheath, representing the inner coaxial sleeve. Note: draining side-holes of the inner sleeve are not visualized in this photograph.

FIG. 8 is a schematic drawing of a cross-sectional (long axis) representation of the distal portion of the device in anchored configuration.

FIG. 9 is a schematic drawing of a color-coded cross-sectional (long axis) representation of the device in anchored configuration.

FIG. 10 is a schematic drawing of a close-up cross-sectional (long axis) diagram of the distal end of the catheter in anchored configuration. The inner and outer coaxial sleeves are permanently attached to each other at their distal ends, around their circumference, forming the distal end-hole (not pictured: drainage side-holes in the inner sleeve).

FIG. 11 is a schematic drawing of a cross-sectional diagram of the distal portion of the device in anchored configuration. Drainage side-holes of the inner coaxial sleeve are depicted and labeled as such.

FIG. 12 is a schematic drawing of a cross-sectional diagram of the distal portion of the device in anchored configuration with detailed, three-dimensional depiction of the portion of the inner coaxial sleeve containing the drainage side-holes.

FIG. 13 is a schematic drawing of a cross-sectional diagram of the distal portion of the device in anchored configuration with detailed, three-dimensional depiction of the portion of the inner coaxial sleeve containing the drainage side-holes.

FIG. 14 is a schematic drawing of a diagram depicting the parts for the repair kit. The luer, representing the needleless valve design. The cap representing the attaching site of the luer lock when conducting the repair.

FIG. 15 is a schematic drawing of the following steps for repairing a damaged nephrostomy tube at the site of the hub. (1) shows a damaged hub depicted by the dotted line, a common location for nephrostomy tube damage. (2). The first step in the repair process is to clamp the nephrostomy tube proximal to the site of damage and cutting the nephrostomy tube proximal to the damage site, yet distal to the clamp to prevent flow of fluid.

FIG. 16 is a schematic drawing of the next step which is to insert the cap into the nephrostomy tube. The cap will slide over the nephrostomy tube as depicted. (4) The next step is to screw in the new needleless valve into the cap.

FIG. 17 is a schematic drawing of a finally, when the clamp can be removed so that the nephrostomy tube can be used.

### DESCRIPTION OF THE PREFERRED EMBODIMENT

In the prior art percutaneous nephrostomy catheter system, the prior art percutaneous nephrostomy catheter system includes a nephrostomy catheter lumen, an extension tubing, a collection bag, a 3-way stopcock assembly and a catheter hub. The primary function of a 3-way stopcock assembly is to enable flushing of the catheter lumen without disconnecting the extension tubing from the collection bag. Unfortunately, the 3-way stopcock assembly and the catheter hub are bulky and uncomfortable, partly contributing to decreased quality of life for patients.

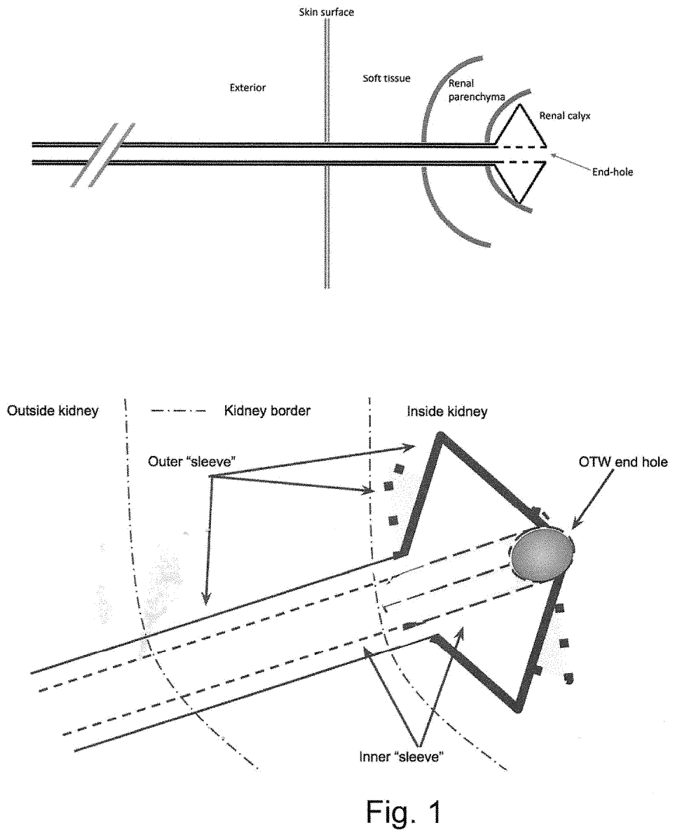

Referring to FIG. 1 an improved percutaneous nephrostomy catheter system 10 provides refinements in its design which center around the primary goals of 1) improving patient comfort/user friendliness and 2) enabling simple, lower risk and inexpensive clinic or bedside repair in the case of fracture of the external catheter. The improved percutaneous nephrostomy catheter system 10 includes a nephrostomy catheter lumen 11, an extension tubing 12, a collection bag 13, and catheter hub 15 to facilitate nephrostomy care while eliminating the need for the 3-way stopcock assembly. When the percutaneous nephrostomy system 10 is in place urine flows from the renal pelvis into the nephrostomy catheter lumen 11 and into the extension tubing 12 and the collection bag 13.

Still referring to FIG. 1 a retention system employs coaxial sleeves with radial deployment of the distal outer sleeve for renal anchoring mechanism over the wire (OTW).

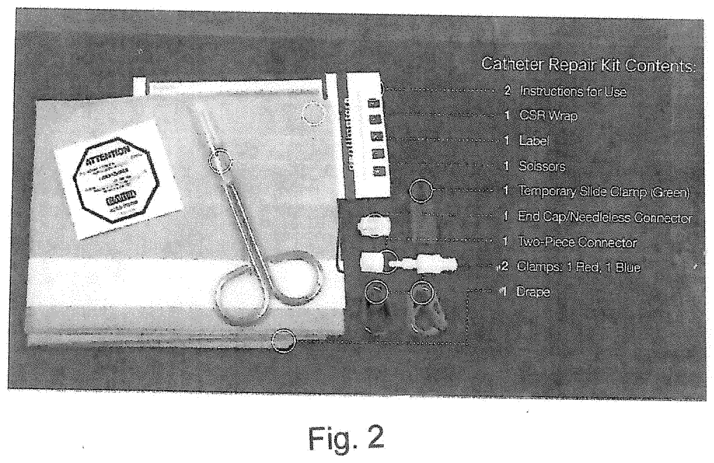

Referring to FIG. 2 is an end cap needleless connector and two-piece connector represent complete hub assembly from a standard venous access catheter repair kit.

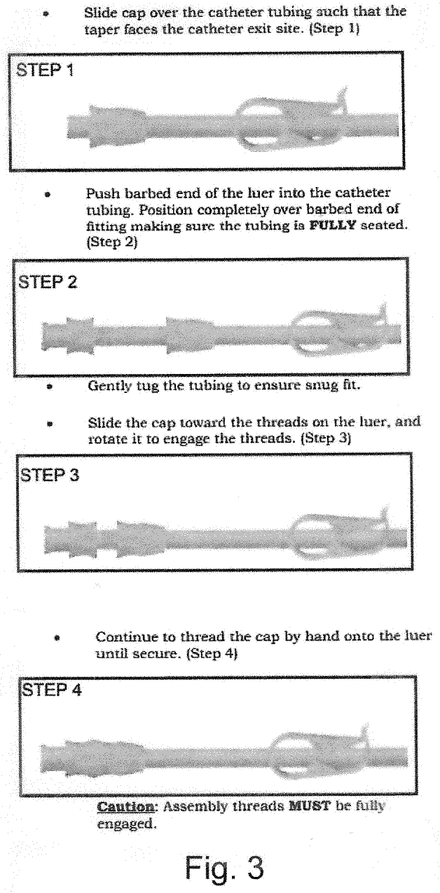

Referring to FIG. 3 a four-step procedure for attaching Medcomp two-part adaptor to catheter tubing.

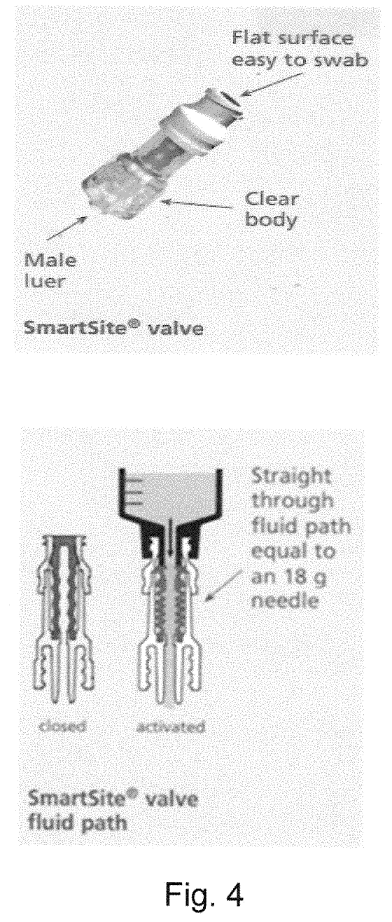

Referring to FIG. 4 is a Luer-activated needleless valve which connects to hub of nephrostomy catheter.

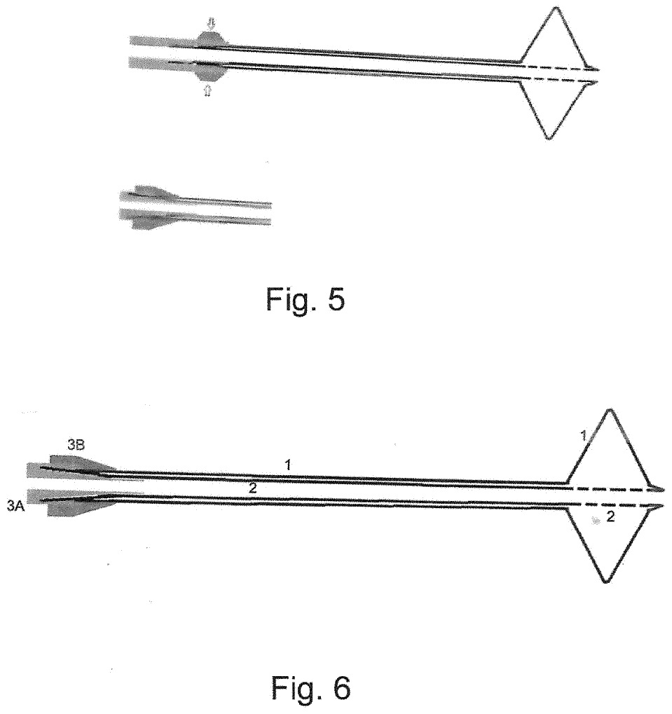

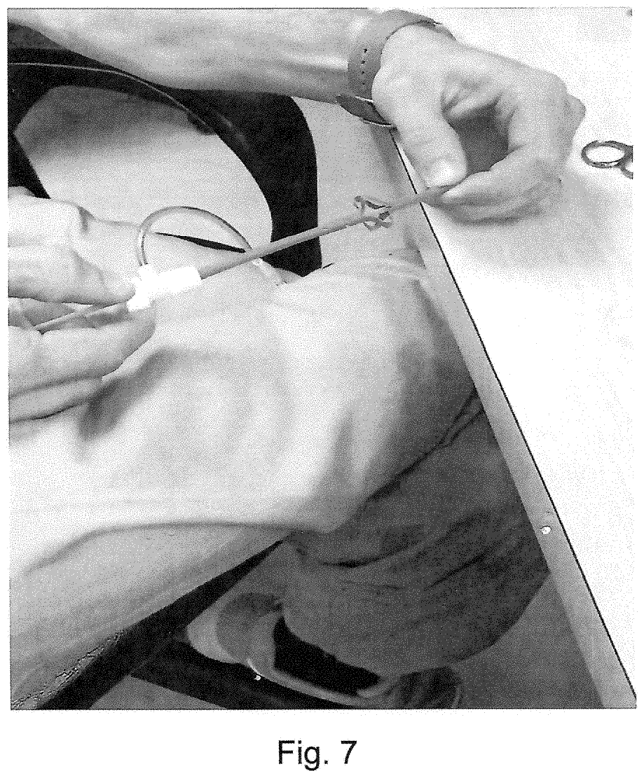

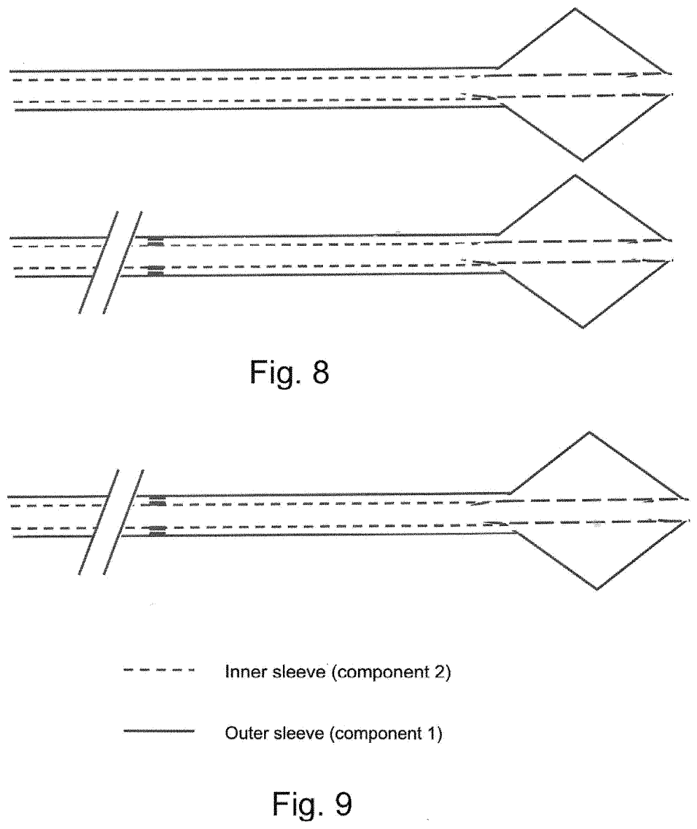

Referring to FIG. 5 in conjunction with FIG. 1 the valve employs a proprietary scalloped Luer-style male connector which, when screwed into the hub 14 allows flow. When the extension tubing 12 is attached to the catheter hub 14, urine flows from the nephrostomy catheter into the collection bag 13. In preparation for flushing the improved percutaneous nephrostomy catheter system 10, a scalloped Luer-style male adaptor is attached to the standard saline flush syringe and the adaptor-syringe combination is screwed into the catheter hub 14. As with the extension tubing 12, the scalloped male connector compresses the valve allowing flow. When connected to the proprietary scalloped male connector, the valve design allows for bidirectional flow.

Referring to FIG. 4 in conjunction with FIG. 2 catheter repair involves cutting off the damaged section and installing a new hub. This process would also cut the retention suture holding the pigtail shape. The improved percutaneous nephrostomy catheter system 10 employs a retention system which is similar to the coaxial Malecot catheter design with some key differences. Actuation of the retention system into the expanded “fixation” position, versus the collapsed “introduction or removal” position, depends on the relative position of the two coaxial sleeves that make up the catheter. The outer sleeve has several longitudinal perforations located around the circumference of its distal end, forming struts oriented radially. The inner sleeve is reinforced and contains multiple side holes in the same region of the longitudinal perforations of the outer sleeve. The two sleeves are fused at the distal end-hole. To prevent formation of a tissue bridge which can hinder removal, our proposed design includes a thin, flexible synthetic membrane, either silicone or ePTFE, forming a partial web over the angles between the proximal struts. Following advancement into the renal pelvis, the outer sleeve is driven toward the patient and the inner sleeve is maintained static, causing the struts of the outer sleeve to flare outwards radially in an inverted “V” shape and the sleeves can be clamped in order to maintain their position relative to one another. The catheter (both inner and outer sleeves) is cut to a custom length chosen according to the specific ergonomic needs of the patient. At this point the hub containing the spring-loaded ball valve within the female end of the Luer-style connection is fastened to the catheter employing a system in a similar fashion to repairable venous access devices or implantable infusion ports. Repair of the nephrostomy catheter simply involves clamping the catheter with padded or atraumatic hemostat or clamp to secure “deployed” shape of the retention system within the renal pelvis, cutting the catheter proximal to the site of damage, and attaching a new hub as described above. The two-piece connector would serve dual duty: 1) connecting the catheter tube to the valve component; and 2) holding the two catheter sleeves in their relative positions in order to maintain the “deployed” configuration of the distal anchoring mechanism (the “Malecot-style” system).

Referring to FIG. 9 a color-coded cross-sectional (long axis) representation of the device is shown in anchored configuration.

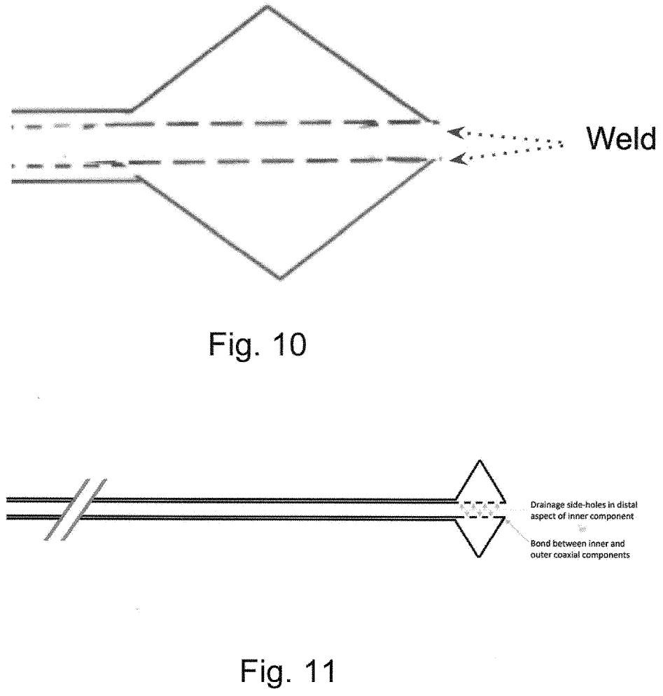

Referring to FIG. 10 a close-up cross-sectional (long axis) diagram of the distal end of the catheter is shown in anchored configuration. The inner and outer coaxial sleeves are permanently attached to each other at their distal ends, around their circumference, forming the distal end-hole (not pictured: drainage side-holes in the inner sleeve).

Referring to FIG. 11 a cross-sectional diagram of the distal portion of the device is shown in anchored configuration. Drainage side-holes of the inner coaxial sleeve are depicted and labeled as such.

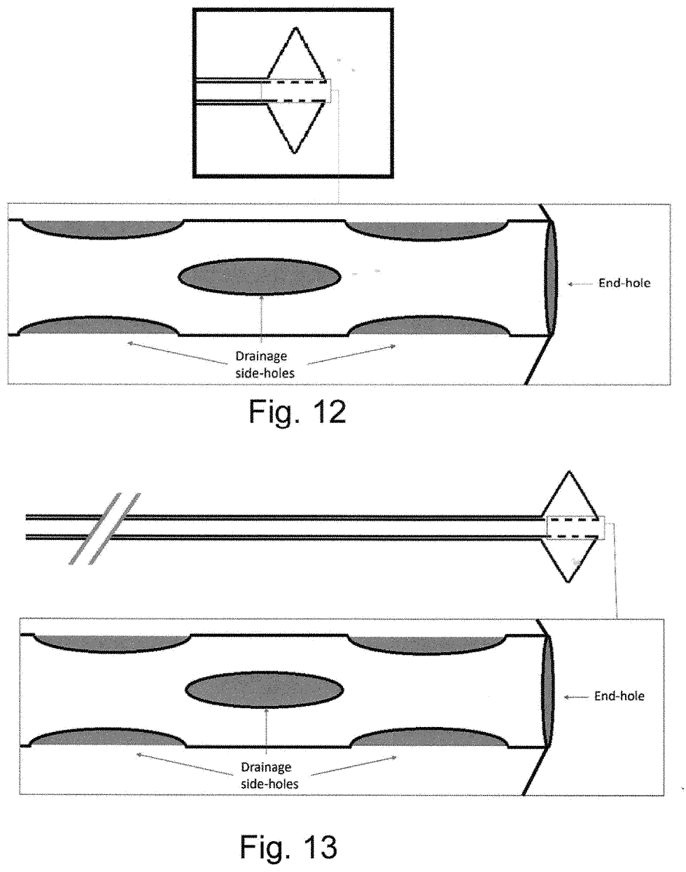

Referring to FIG. 12 a cross-sectional diagram of the distal portion of the device is shown in anchored configuration with detailed, three-dimensional depiction of the portion of the inner coaxial sleeve containing the drainage side-holes.

Referring to FIG. 13 a cross-sectional diagram of the distal portion of the device is shown in anchored configuration with detailed, three-dimensional depiction of the portion of the inner coaxial sleeve containing the drainage side-holes.

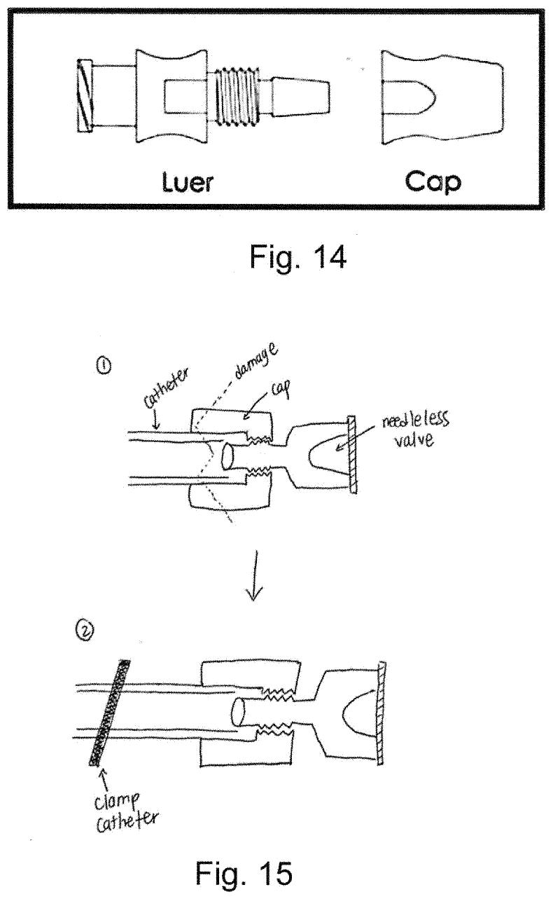

Referring to FIG. 14 a diagram depicts the parts for the repair kit. The luer represents the needleless valve design. The cap represents the attaching site of the luer lock when conducting the repair.

Referring to FIG. 15 the following steps for repairing a damaged nephrostomy tube at the site of the hub are as follows: (1) shows a damaged hub depicted by the dotted line, a common location for nephrostomy tube damage. (2). The first step in the repair process is to clamp the nephrostomy tube proximal to the site of damage and cutting the nephrostomy tube proximal to the damage site, yet distal to the clamp to prevent flow of fluid.

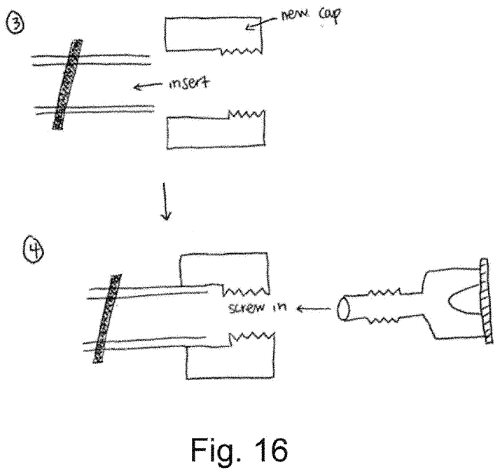

Referring to FIG. 16 the next step is to insert the cap into the nephrostomy tube. The cap will slide over the nephrostomy tube as depicted. (4) The next step is to screw in the new needleless valve into the cap.

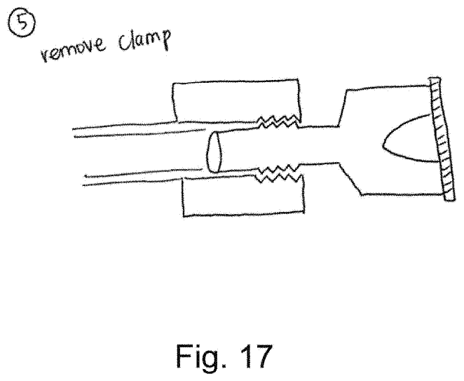

Referring to FIG. 17 when a clamp can be removed then the nephrostomy tube can be used.

This ubiquitous device utilizes a silicone valve seal which opens upon insertion of any standard male Luer-lock fitting. Some manufacturers recommend replacement of these valves every three days, this is based upon usage as an intravenous infusion fitting. Testing would be required to elucidate the appropriate replacement interval for these valves in the setting of urinary diversion. Luer-activated needleless valves may represent a solution with very low development and manufacturing cost.

The improved percutaneous nephrostomy system 10 incorporates design elements used in percutaneous catheters, e.g. Malecot nephrostomy and suprapubic catheters, repairable hemodialysis catheters and luer-activated valves. Regarding outside diameter of the catheter relative to the luminal diameter, the coaxial design would sacrifice no more than 1-2 French of luminal diameter. Both versions of the valve design, the novel ball-spring-valve and the existing luer-activated valve, represent low cost solutions to significant problems of patient quality of life and repairability of percutaneous nephrostomy. These refinements in this percutaneous nephrostomy catheter design center around the primary goals of 1) improving patient comfort/user friendliness, 2) improving stability against dislodging, and 3) enabling simple, lower risk, and inexpensive clinic or bedside repair in the case of fracture of the external catheter. The stopcock facilitates flushing without leakage while adding to the bulk of the external portion of the drainage system, negatively impacting patient comfort and QoL. A thin-walled, large lumen Luer-activated valve may be inserted onto the hub. The Luer-activated valves prevent leakage upon disconnecting the extension tubing during bag emptying, flushing and allows for bi-directional flow upon insertion of Luer-lock counterpart. The current Luer-activated valves are ubiquitous in the setting of venous access catheters and IV tubing (infusion of drugs) current valves have a luminal diameter equivalent to that of an 18-gauge needle (0.84 mm) while typical 8 Fr nephrostomy catheters have a luminal diameter of 1.7 mm. This 50% bottleneck would create flow restriction if used in nephrostomy systems, especially in patients with viscous urine. A large-bore Luer-activated design with unique internal construction allowing 1.7-2 mm of luminal diameter is recommended. The locking pigtail designs are the current standard in nephrostomy and other percutaneous drainage catheters. The design is not without issues. The pigtail conformation of the distal catheter depends on a thin string. Failure of this retention string is not uncommon, leading to catheter displacement a hospital visit for replacement/exchange. This design totally precludes any repair of external catheter fracture (i.e. near hub), as cutting the catheter would transect the retention string. The solution is a coaxial radial retention system. The catheter is made up of two coaxial “sleeves” fused at the distal end hole. The distal part of the outer sleeve has several (4-8) linear, longitudinal perforations, which upon actuation of the retention system create several V-shaped retention struts oriented radially around the axis of the catheter. The inner sleeve contains multiple side holes in the same region of the longitudinal perforations of the outer sleeve and is reinforced to provide support for the retention struts. This is not a Malecot-style catheter. Malecot catheters rely on tension in the pre-bent struts and contains no coaxial support component. The only thing in common with our design is the radial orientation of V-shaped struts. Actuation of the retention system into the expanded “fixation” position versus the collapsed “introduction or removal” position depends on the relative position of the two coaxial sleeves that make up the catheter. Once advanced into the renal pelvis, the outer sleeve is driven toward the patient and the inner sleeve is maintained static, causing the struts of the outer sleeve to flare outwards and assumed their V-shape. The hub design allows the catheter tip to be locked in this configuration. The two components of the hub are the main hub with a male nozzle fitted and welded to inside of inner sleeve from factory with the sleeve flared outward around male nozzle to preserve luminal diameter and the male threads on external surface of main hub to accept outer collar and the outer collar (compression-collar) with female thread to fasten onto main hub, effectively compressing the two sleeves together a fixation of distal catheter in “retention conformation.” The outer collar is supplied free-floating around the outer sleeve. This design also allows repair in case of external catheter fracture by simply clamping the catheter with padded or atraumatic hemostat or clamp to secure “deployed” shape of the retention system within the renal pelvis, cut the catheter proximal to the site of damage and attach a new hub as described previously. If there are concerns of tissue bridge formation hindering full collapse for removal, our proposed design could include a thin, flexible synthetic membrane, either silicone or urethane, forming a web over the parts of the struts adjacent to the calyx wall.

From the foregoing it can be seen that an improved percutaneous nephrostomy system has been described. It should be noted that the sketches are not drawn to scale and that distances of and between the figures are not to be considered significant.

Accordingly, it is intended that the foregoing disclosure and showing made in the drawing shall be considered only as an illustration of the principle of the present invention.

### REFERENCES

1. Dagli, M., & Ramchandani, P. (2011). Percutaneous Nephrostomy: Technical Aspects and Indications. Seminars in Interventional Radiology, 28(04), 424-437. doi:10.1055/s-0031-1296085
2. Fingar, K. R., Stocks, C., Weiss, A. J., & Steiner, C. A. (2014, December). Most Frequent Operating Room Procedures Performed in U.S. Hospitals, 2003-2012. Retrieved Feb. 2, 2019, from https://www.hcup-us.ahrq.gov/reports/statbriefs/sb186-Operating-Room-Procedures-Unit ed-States-2012.jsp
3. Percutaneous Nephrostomy. (2018, February 26). Retrieved Feb. 7, 2019, from https://www.dovemed.com/common-procedures/procedures-surgical/percutaneous-nephrostomy/
4. Nephrostomy Catheter Placement. (2019). Retrieved Feb. 7, 2019, from https://www.mdsave.com/procedures/nephrostomy-catheter-placement/d581facb
5. Noureldin, Y. A., Diab, C., Valenti, D., & Andonian, S. (2016). Circle nephrostomy tube revisited. Canadian Urological Association Journal, 10(7-8), 223. doi:10.5489/cuaj.3596
6. Assimos, D. (2015 Jul. 1). Re: Determinants of Nephrostomy Tube Dislodgment after Percutaneous Nephrolithotomy. Retrieved from https://insights.ovid.com/pubmed?pmid=26088231#
7. Nephrostomy. (2017 Jul. 26). Retrieved from https://www.insideradiology.com.au/nephrostomy/
8. Bayne, D., Taylor, E. R., Hampson, L., Chi, T., & Stoller, M. L. (2015). Determinants of nephrostomy tube dislodgment after percutaneous nephrolithotomy. Journal of endourology, 29(3), 289-92.
9. Sarkar, D., & Parr, N. J. (2018 Jul. 1). Dislodged nephrostomy: A top tip. Retrieved from https://innovations.bmj.com/content/4/3/113
10. Lewis, S., & Patel, U. (2004, February). Major complications after percutaneous nephrostomy-lessons from a department audit. Retrieved from https://www.ncbi.nlm.nih.gov/pubmed/14746788
11. Cielli, C. (2019 January 28). Insufficient sleep: Definition, epidemiology, and adverse outcomes. Retrieved Feb. 7, 2019, from https://www.uptodate.com/contents/insufficient-sleep-definition-epidemiology-and-adverse-outcomes?search=sleep quality&source=search_result&selectedTitle=1˜150&usage_type=default& display_rank

What is claimed is:

1. An improved percutaneous nephrostomy catheter system 10 comprising:
a. a nephrostomy catheter lumen;
b. an extension tubing inserted into said nephrostomy catheter lumen;
c. a collection bag fluidly coupled to said extension tubing;
d. a catheter hub which employs a Luer valve in order to facilitate nephrostomy care so that when said percutaneous nephrostomy system is in place urine flows from the renal pelvis into said nephrostomy catheter lumen, said extension tubing and said collection bag.

2. An improved percutaneous nephrostomy catheter system according to claim 1 wherein said valve employs a scalloped Luer-style male connector which, when screwed into said catheter hub allows flow.
